(19) United States
(12) Patent Application Publication
(10) Pub. No.: US 2026/0234601 A1
(43) Pub. Date: Aug. 13, 2026

(54) NOVEL POROUS CARBONACEOUS MATERIAL, PROCESS FOR THE PREPARATION THEREOF, AND USES THEREOF

(71) Applicants: CENTRE NATIONAL DE LA RECHERCHE SCIENTIFIQUE, PARIS (FR); UNIVERSITE DE MONTPELLIER, MONTPELLIER (FR); ÉCOLE NATIONALE SUPERIEURE DE CHIMIE DE MONTPELLIER, MONTPELLIER CEDEX 5 (FR)

(72) Inventors: Jullien DRONE, MONTPELLIER (FR); Nicolas BRUN, PRADES LE LEZ (FR); Coline MATEOS, MONTPELLIER (FR)

(73) Assignees: CENTRE NATIONAL DE LA RECHERCHE SCIENTIFIQUE, PARIS (FR); UNIVERSITE DE MONTPELLIER, MONTPELLIER (FR); ÉCOLE NATIONALE SUPERIEURE DE CHIMIE DE MONTPELLIER, MONTPELLIER CEDEX 5 (FR)

(21) Appl. No.: 19/148,029

(22) PCT Filed: Jan. 15, 2024
(86) PCT No.: PCT/EP2024/050824

§ 371 Date: Jul. 15, 2025

(30) Foreign Application Priority Data
Jan. 16, 2023 (FR) FR2300391

(51) Int. Cl. C12N 11/089; C08L 73/00

(52) U.S. Cl. CPC C12N 11/089; C08L 73/00; C12Y 103/01031; C12Y 206/01077; C12Y 306/01001

(57) ABSTRACT

The present invention relates to a carbonaceous polymeric material also referred to as porous carbonaceous material complexed with at least one metal ion, preferably a metal cation, a composition comprising a protein immobilized on said material, and methods of preparing the same. The invention further relates to the use of the composition comprising an immobilized protein in particular in heterogeneous biocatalysis applications.

### FIELD

The present invention relates to a carbon-based polymeric material also called porous carbon-based material, or carbon-rich aerogel, comprising at least one complexed metal ion, preferably a metal cation, and a composition comprising at least one protein immobilized on said material and methods for preparing the same.

The invention also relates to the use of the composition comprising at least one immobilized protein in heterogeneous biocatalysis applications in particular.

A method for immobilizing proteins bearing an affinity marker on said porous carbon-based material is also provided.

### BACKGROUND

Porous carbon-based materials or carbon-rich aerogels, known from the state of the art, are conventionally obtained by sol-gel polymerization reactions between carbon-rich organic precursors and drying of the resulting hydrogels.

A porous carbon-based material of the type prepared and used in this application is described in the publication “Hydrothermal synthesis of highly porous carbon monoliths from carbohydrates and phloroglucinol”; Nicolas Brun et al.; RSC Advances, 2013, 3.

In particular, this paper describes an original hydrothermal approach to synthesize highly porous carbon-rich cryogels and aerogels (or carbogels) from carbohydrate-based precursors (e.g. fructose, glucose or xylose) and phenolic compounds (e.g. phloroglucinol). It should be mentioned that these carbohydrates can be isolated from the cellulosic fraction of the lignocellulosic biomass, while phloroglucinol is the monomer unit of phlorotannins and can be isolated from the bark of fruit trees, brown algae or by various biosynthetic pathways. Therefore, the compounds used in this patent application can be considered renewable and sustainable.

State-of-the-art carbon-rich aerogels derived from biomass are obtained after further heating treatment at a temperature above 300° C. and offer a wide range of potential applications, such as electrodes for batteries and fuel cells, or adsorbents for hydrogen and carbon dioxide storage. The carbon-rich aerogels or porous carbon-based materials according to the present invention are not subjected to heating treatment at a temperature ≥300° C.

In addition, enzymes are a specific group of proteins that serve as biological catalysts in the metabolism of all living cells. Thus, enzymes are able to specifically transform organic and inorganic molecules into products of interest.

However, since enzymes are biological molecules developed for one cellular environment, they are often unsuitable for other environments. It is therefore interesting to be able to immobilize enzymes on a solid support and use them as catalysts in this immobilized state.

Solid-based enzyme immobilization was performed using different techniques and different solid supports. Adsorption of enzymes on solid surfaces can lead to undesirable interactions between the enzyme and the solid support. For example, it has been shown that adsorption of proteins on silica nanoparticles can lead to changes in the secondary structure of the protein, which can lead to deactivation of the enzyme. It is therefore important that the solid support does not interfere with the structure and activity of the immobilized enzymes.

Immobilized metal ion affinity chromatography (IMAC) is a protein purification technique based on the affinity of proteins carrying a poly-histidine sequence for metal ions such as Fe³⁺, Co²⁺, Zn²⁺, Cu²⁺, or Ni²⁺.

Thus, for example, metal ions are immobilized on an agarose gel carrying IDA (imido diacetate) and/or NTA (nitrilo triacetate) functions and can be selectively complexed by amino acid-carrying proteins such as histidine and/or cysteine. An improved version of this technique uses recombinant proteins containing a fused polyhistidine peptide. Although this technique can be successfully applied in chromatographic procedures for the purification and isolation of proteins, enzymes immobilized on a gel are less suitable as heterogeneous catalysts in organic synthesis.

In attempts to prepare heterogeneous catalysts, the IMAC-based principle of affinity marker binding was applied to the immobilization of polyhistidine-labeled enzymes on modified silica. Encouraging results were obtained for Candida antarctica lipase B (CalB), but other less stable enzymes were found to be deactivated in the presence of silica, especially in the presence of organic solvents. It is known in the literature that silica nanoparticles have a destabilizing effect on proteins.

The high costs of enzyme preparation and the loss of activity frequently observed when immobilizing the enzyme on a solid support are obstacles in this development. A standardized, generally usable procedure for enzyme immobilization, which would allow the enzyme to be reused, would be highly desirable. There is still no general and simple method for the preparation of heterogeneous catalysts by immobilization of enzymes.

There is therefore a need for new supports and improved methods for immobilizing enzymes on these supports, and for stable heterogeneous biocatalysts that can be applied in organic synthesis under conditions of aqueous or organic reactions.

Surprisingly, it has been discovered that a porous carbon-based material obtained by hydrothermal carbonization of at least one carbohydrate and at least one compound carrying at least one orthodiphenol function, charged with at least one metal ion, can immobilize at least one protein, preferably via affinity tag binding, in an improved way compared to other types of protein immobilization.

### SUMMARY

In this context of a search for suitable and efficient biocatalysis tools, a first purpose of the invention is to propose a porous carbon-based material complexed with at least one metal ion, preferably a metal cation. A second purpose of the invention is to propose a composition comprising a protein immobilized on said material or the use of said material to immobilize a protein. A third purpose of the invention is to propose methods for preparing said materials and compositions. Finally, another purpose of the invention is to propose particular compositions and their uses.

### BRIEF DESCRIPTION OF FIGURES

FIG. 1 is a scanning electron micrograph of a continuously interconnected uniform porous texture for a tannin-xylose aerogel prepared in 10 mL of a 15% wt-dry solution of absolute ethanol in demineralized water, treated at 180° C. for 20 hours under hydrothermal conditions (autoclave), washed and then dried by freeze-drying.

FIG. 2 is a transmission electron micrograph that shows a macro-mesoporous network for a tannin-xylose aerogel prepared in 10 mL of a 15% solution by mass of absolute ethanol in demineralized water, treated at 180° C. for 20 hours under hydrothermal conditions (autoclave), washed and then dried by freeze-drying.

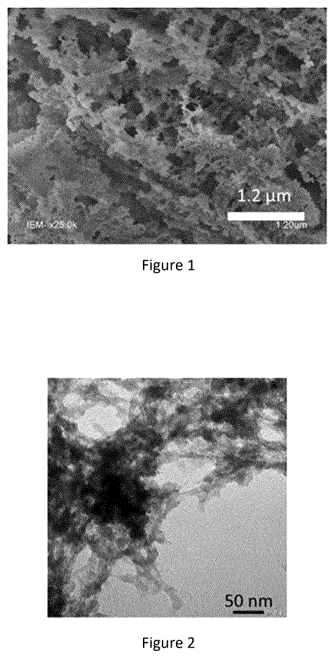

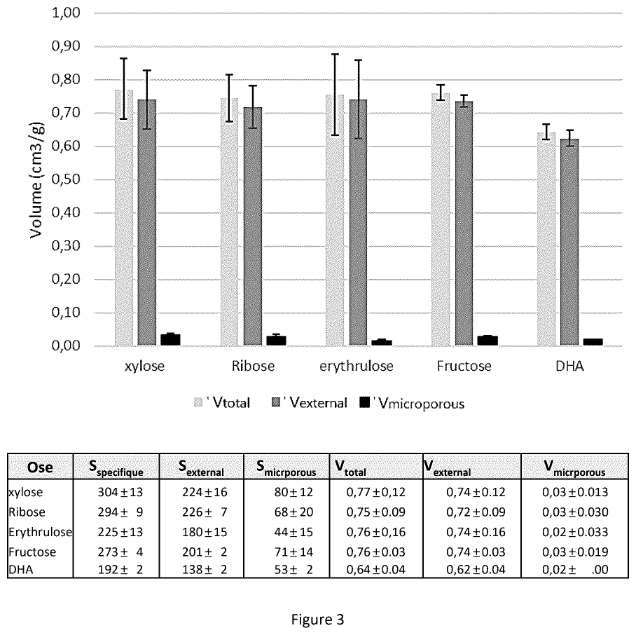

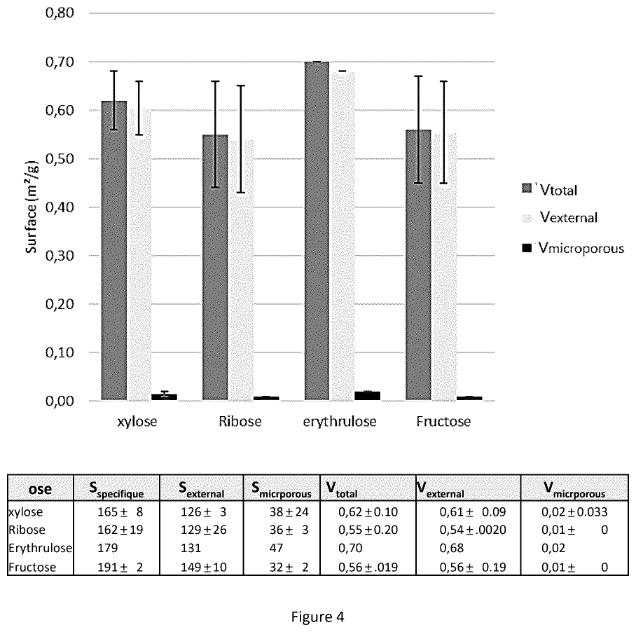

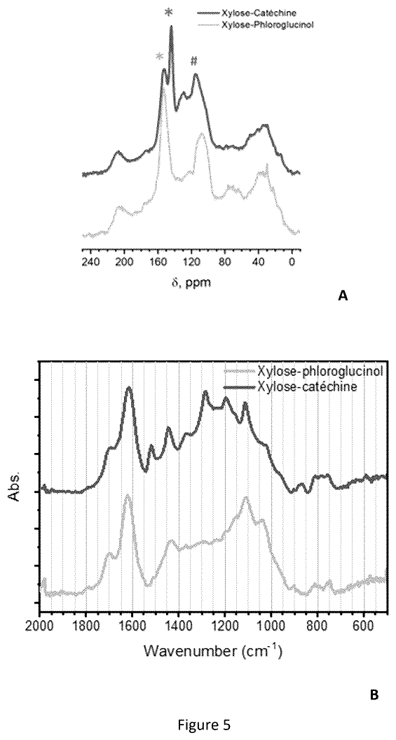

FIGS. 1 and 2 show primary particles of a few nanometers, aggregated primary particles forming an interconnected network that induces interparticle interstices of a few nanometers to several micrometers.

FIG. 3 represents the textural properties determined by volumetry by nitrogen sorption to 77K of aerogel prepared from a mixture of an ose and natural catechu extract.

FIG. 4 shows the textural properties determined by volumetrics by sorption of nitrogen to 77K of aerogel prepared from a mixture of an ose and catechin.

FIG. 5A represents a 13C CP-MAS NMR spectrum of an aerogel formed by hydrothermal carbonization of a xylose-catechin (top, dark) and xylose-phloroglucinol (bottom, light) mixture.

FIG. 5B an FTIR spectrum of an aerogel formed by hydrothermal carbonization of a xylose-catechin (top, dark) and xylose-phloroglucinol (bottom, light) mixture.

### DETAILED DESCRIPTION

According to a first aspect of the invention, the present application relates to a composition comprising:
(a) a porous carbon-based material obtained by hydrothermal carbonization comprising or formed from at least one carbohydrate, preferably at least one ose, or at least one polysaccharide (bio-based precursor #1), and
   at least one compound with an orthodiphenol or orthomethoxyphenol function (bio-based precursor #2), and
(b) at least one metal cation.

The porous carbon-based material of the present invention is characterized with 13CP-MAS solid-state NMR spectroscopy by a signal at 144 ppm, attributed to the carbons of the orthodiphenol functions.

The porous carbon-based material of the present invention is also characterized by bands at 1200, 1280 and 1500 cm⁻¹, in FTIR spectroscopy, attributed to the C—C and C—O vibrations of the aromatic ring of an orthodiphenol function.

The porous carbon-based material of the present invention therefore has at least one orthodiphenol function.

The porous carbon-based material of the present invention is obtained by hydrothermal carbonization of at least one carbohydrate, preferably at least one ose, or at least one polysaccharide (bio-based precursor #1) and at least one compound with an orthodiphenol or orthomethoxyphenol function (bio-based precursor #2).

The bio-based precursor compound #1 usable according to the present invention is a carbohydrate, preferably at least an ose, or at least a polyoside.

Carbohydrates are defined by the International Union of Pure and Applied Chemistry (IUPAC) as a class of organic compounds containing a carbonyl group (aldehyde or ketone) and at least two hydroxyl groups (—OH). This class includes substances derived from monosaccharides by reduction of the carbonyl group, oxidation of at least one functional group at the end of the chain to carboxylic acid, or replacement of one or more hydroxyl groups by a hydrogen atom, an amino group, a thiol group or any similar atom. In addition, compounds derived from the dehydration of carbohydrates (e.g. furan aldehydes) may also be used in the context of the present application.

An ose (or monosaccharide) is a carbohydrate monomer. Oses have at least 3 carbon atoms: they are polyhydroxyaldehydes or polyhydroxyketones.

Oses are distinguished by the length of their carbon chain, as follows:
trioses: 3-carbon oses, C₃H₆O₃ (glyceraldehyde, dihydroxyacetone);
tetroses: 4-carbon oses, C₄H₈O₄ (erythrose, threose, erythrulose);
pentoses: 5-carbon oses, C₅H₁₀O₅ (deoxyribose (C₅H₁₀O₄), ribose, arabinose, xylose, lyxose, ribulose, xylulose)
hexoses: 6-carbon oses, C₆H₁₂O₆ (allose, altrose, galactose, glucose, gulose, idose, mannose, talose, fructose, psicose, sorbose, tagatose);
deoxyhexoses: 6-carbon oses, C₆H₁₂O₅ (fucose, rhamnose);
heptoses: 7-carbon oses, C₇H₁₄O₇ (sedoheptulose, mannoheptulose)
the octoses: 8-carbon oses, C₈H₁₆O₈ (heptahydroxyoctanal);
nonoses: 9-carbon oses, C₉H₁₇N₁O₈ (neuraminic acid or sialic acid).

An aldose is a monosaccharide consisting of a chain of n carbon atoms with a carbonyl group on the terminal carbon atom, making it an aldehyde, and hydroxyl groups connected to all other carbon atoms. Aldoses have the general chemical formula Cₙ(H2O)ₙ. Since formaldehyde (n=1) and glycolaldehyde (n=2) are generally not considered carbohydrates, the simplest possible aldose is glyceraldehyde (n=3), which contains only three carbon atoms.

A ketose is a monosaccharide consisting of a chain of n carbon atoms with a carbonyl group on the non-terminal carbon atom, making it a ketone, and hydroxyl groups connected to all other carbon atoms.

According to a preferred mode of invention, the at least one ose is chosen from:
an aldose chosen from: glyceraldehyde, erythrose, threose, ribose, arabinose, xylose, lyxose, allose, altrose, glucose, mannose, gulose, idose, galactose, and talose, preferably xylose,
   or
a ketose chosen from: dihydroxyacetone, erythrulose, ribulose, xylulose, fructose, psicose, sorbose, and tagatose, preferably fructose

A polysaccharide is a polymer of the carbohydrate family formed by an n number of bones (n≥2) by alpha or beta glycosidic bonding.

According to a preferred mode of the invention, the at least one polysaccharide is chosen from:
a heterodiholoside (a polysaccharide for which n=2 consists of 2 different oside units) chosen from: trehalulose, sucrose, turanose, maltulose, leucrose, isomaltulose, gentiobiulose, melibiose, lactulose, lactose, and rutinose, or
a homodiholoside (a polysaccharide for which n=2 consists of 2 identical oside units) chosen from: inulobiose, alpha2-mannobiose, alpha3-mannobiose, trehalose, kojibiose, nigerose, maltose, isomaltose, sophorose, laminaribiose, cellobiose, and gentiobiose, preferably maltose or sucrose.

According to another mode of the invention, the bio-based precursor compound #1 of the porous carbon material consists of a carbohydrate, preferably an ose, or a polysaccharide as described above.

According to another mode of the invention, the porous carbon material comprises two bio-based precursor compounds #1 which consist of two carbohydrates, preferably two oses, two polysaccharides or one ose and one polysaccharide as described above. In another mode of invention, the porous carbon material comprises more than two #1 bio-based precursor compounds.

According to a preferred mode of the invention, the at least one compound having an orthodiphenol or orthomethoxyphenol function is a bio-based precursor compound having at least one benzene ring substituted by two hydroxyl groups in the ortho position or at least one methoxy group in the ortho position.

In this application, the term “bio-based” refers to materials or compounds derived from biomass of plant or animal origin, whether chemically modified or not. These raw materials have the advantage of being renewable.

A compound “of natural origin” or bio-based within the meaning of the invention means any compound that comes from nature (biomass of plant or animal origin). This compound can be extracted from renewable terrestrial and marine biomass or from living organisms (animals, micro-organisms), possibly subsequently modified, for example chemically, or obtained following the action of living micro-organisms (e.g. enzymes or bacteria) on a naturally occurring compound using methods such as biofermentation or biosynthesis. Petroleum-based compounds, of fossil origin, do not fall into this category.

The bio-based precursor compound #2 usable according to the present invention may be a compound having at least one orthodiphenol function.

The compound having at least one bio-based orthodiphenol or precursor orthomethoxyphenol function usable according to the present invention can be selected from among the secondary metabolites of plants. Phenolic compounds form the most important group of phytochemicals in plants. They present nearly 8000 molecules divided into a dozen chemical classes. Each class is characterized by the presence of a benzene ring to which one or more hydroxyl groups are directly bound. These compounds are present in all parts of higher plants (roots, stems, leaves, flowers, pollen, fruits, seeds and wood). They are synthesized by plants subjected to harsh conditions (infections, injuries, UV radiation, etc.) and are involved in many physiological methods such as cell growth, rhizogenesis, seed germination or fruit ripening.

The preferred orthodiphenol or orthomethoxyphenol compounds are listed in the Table below:

| Carbon | | |
|---|---|---|
| skeleton | Class | Example |
| C₆ | Simple phenols | Catechol, pyrogallol |
| C₆-C₃ | Hydroxycinnamic acids | Caffeic acid |
| C₆-C₁-C₆ | Xanthones | Mangiferine |
| C₆-C₃- | Flavonoids, Flavonols, | Quercetin, cyanidin, Catechin, |
| C₆ | Anthocyanes, Flavanols | epicatechin |
| | Flavanones | |
| (C₆-C₃- | Condensed tannins | anthocyanidol, procyanidol B-3, |
| C₆)ₙ | | prodelphinidol B-4 |
| | Phlorotannin | fuhahols (bifuhalol, trifuhalol) |
| | | carmalol |
| | | (diphloréthohydroxycamanol) |
| | Tannins hydrolysables | Tannic acid |

According to a preferred mode of the invention, the at least one compound having an orthodiphenol or orthomethoxyphenol function is selected from: catechol, pyrogallol, caffeic acid, mangaferin, quercetin, cyanidin, catechin, epicatechin, epigallocatechin, anthocyanidol, procyanidol B-3, procyanidol B-4, fuhalols (bifuhahol, trifuhahol), carmalol (diphlorethohydroxycamalol) and tannic acid.

According to a preferred mode of the invention, the at least one compound having at least one orthodiphenol function is catechin.

According to a preferred mode of the invention, the at least one compound having at least one orthodiphenol or orthomethoxyphenol function is contained in a natural plant extract.

According to a preferred mode of invention, the natural vegetable extract containing at least one compound having at least one orthodiphenol or orthomethoxyphenol function is selected from an extract of strawberry, potato, apple, lemon, walnut, vine, grape, flowers, soybean, peas, pine, tomato, garlic, quebracho wood, mimosa, chestnut or catechu.

According to another mode of the invention, the porous carbon material comprises two bio-based precursor compounds #2 which consist of two compounds having at least one orthodiphenol function as described above. According to another mode of the invention, the porous carbon material comprises more than two bio-based precursor compounds #2.

According to a preferred mode of the invention, the porous carbon-based material obtained by hydrothermal carbonization comprising or formed from:
maltose, galactose, lactose, glucose, mannose, sucrose, arabinose, ribose, fructose, xylose, erythrose, or dihydroxyacetone, and
catechin, or an extract of catechu.

Catechu extract can be obtained from plant fibres of Acacia catechu, said extract containing condensed tannins and flavonols.

According to the invention, the term “porous carbon-based material” represents a polymeric material obtained or capable of being obtained by sol-gel polymerization (e.g. by hydrothermal carbonization) from at least two bio-based precursors #1 and 2 as defined in the present application.

According to the invention, the molar ratio between the at least one carbohydrate, preferably at least one ose, or at least one polysaccharide and the at least one compound having at least one orthodiphenol or orthomethoxyphenol function is from 10:1 to 1:10, preferably 1/2 or 1/1 or 2/1 or 3/1 or 4/1 or 5/1.

In a mode of embodiment of the invention, the porous carbon-based material is capable of being obtained by a sol-gel polymerization method, known to the skilled person, involving at least two bio-based precursors #1 and 2 as defined above.

Sol-gel polymerization methods can use a catalyst, for example, acids and bases such as, for example, nitric acid, acetic acid, ascorbic acid, hydrochloric acid, sulfuric acid, boric acid, sodium carbonate, sodium hydroxide, ammonium hydroxide, and calcium sulfate.

Catalyst concentrations can be expressed as a reagent-to-catalyst (R/C) ratio, which can range, for example, from 10 to 5,000, or from 10 to 2,000, or from 10 to 1,000.

According to a preferred mode of the invention, the porous carbon-based material is obtained by hydrothermal carbonization.

Hydrothermal carbonization is defined as a method of thermochemical conversion of biomass or, in the context of the present invention, at least two bio-based precursors #1 and 2 as defined above for the purpose of obtaining a porous carbon monolith in the aqueous phase. It is an exothermic method that decreases the oxygen and hydrogen levels of matter mainly through dehydration and decarboxylation reactions. Sol-gel polymerization is achieved by applying temperatures from 120° C. and 250° C. to a mixture of at least two bio-based precursors #1 and 2 as defined above and a solvent, preferably water (usually 10% by mass of the two bio-based precursors #1 and 2).

In a particular embodiment, the porous carbon material is prepared by the following method:
a) Heating a reaction mixture comprising an aqueous solution, and at least two bio-based precursors #1 and 2 as defined above dissolved in the aqueous solution, to a temperature below 300° C. to obtain a solid porous carbon material,
b) Washing the solid porous carbon material obtained by successive soaking in a polar solvent.

The aqueous solution comprises water, and possibly solvents, preferably in quantities miscible with water.

According to a preferred embodiment, the reaction mixture consists of only one liquid phase, namely the aqueous solution, preferably water.

For example, and without limitation, the aqueous solution comprises ethanol, preferably absolute ethanol.

According to a preferred embodiment, the mass fraction of water in an absolute water/ethanol mixture is from 1 and 0.3.

According to a preferred embodiment, the mass fraction of water in a water/absolute ethanol mixture is from 1 and 0.5 when the bio-based precursors #1 and 2 are xylose and catechin, respectively.

Step (a) of the method may also be referred to as the “hydrothermal treating step”.

Thanks to the presence of water (in the aqueous solution) in step (a), the method of preparation of the invention differs for example from pyrolytic methods, for biomass conversion or bio-based precursors #1 and 2, to give a charcoal-like material (typically in the absence of oxygen).

The reaction temperature in the hydrothermal treatment step (a) is preferably <300° C., preferably from 100 to 300° C., even more preferably 120 to 250° C. and most preferably from 160 to 200° C. or preferably 180° C. Reaction temperature is intended to refer to the temperature, more specifically the average temperature, inside the reaction mixture, which can be measured with a thermocouple.

The hydrothermal treatment in the method of preparing the porous carbon-based material of the present invention is preferably carried out in a pressure-resistant reactor, e.g. an autoclave.

There is no specific limitation as to the duration of step (a) in the method of preparing the porous carbon-based material of the invention.

For example, the reaction medium can be introduced into a hermetically sealed mineralization bomb and then placed in a thermostatic chamber at 180° C. for 20 hours.

Step (a) of the method is carried out until the entire contents of the reaction mixture comprising the aqueous solution and bio-based precursors #1 and 2 have gelled or precipitated into porous carbon-based material of the present invention.

In a particular embodiment, the porous carbon material is prepared by the following method:
a) Heating a reaction mixture comprising:
   an aqueous solution consisting of water and absolute ethanol with a mass water fraction from 0.7 to 0.4, and,
   xylose and catechin dissolved in the aqueous solution, at a temperature below 300° C. (preferably 180° C.) to obtain a solid porous carbon material,
b) Washing the solid porous carbon material obtained by successive soaking in a polar solvent.

In a particular embodiment, the porous carbon material is prepared by the following method:
a) Heating a reaction mixture comprising:
   an aqueous solution consisting of water and absolute ethanol with a mass water fraction from 1 to 0.5, and,
   xylose and catechin or a catechu extract dissolved in the aqueous solution, at a temperature below 300° C. (preferably 180° C.) to obtain a solid porous carbon material,
b) Washing the solid porous carbon-based material obtained by successive soaking in a solvent, preferably a polar solvent.

According to a preferred embodiment, the carbon-based material is formed from at least one carbohydrate, preferably an aldose or a ketose, even more preferably at least one heterodiholoside, or homodiholoside (c1) and at least one compound with at least an orthodiphenol or orthomethoxyphenol (c2) function in a molar ratio (c1/c2) from 10:1 to 1:10, preferably 2/1. For complex natural extracts and condensed tannins, the molar ratio (c1/c2) is defined as the ratio between the number of moles of carbohydrates (c1) and the number of moles of compounds with at least one orthodiphenol or orthomethoxyphenol function.

In a particular embodiment, the porous carbon material is prepared by the following method:
a) Heating a reaction mixture comprising:
   an aqueous solution consisting of water and absolute ethanol with a mass water fraction from 0.7 to 0.4, and,
   xylose and catechin or a catechu extract dissolved in the aqueous solution, at a temperature below 300° C. (preferably 180° C.) to obtain a solid porous carbon material,
b) Washing the solid porous carbon-based material obtained by successive soaking in a solvent, preferably a polar solvent.

In a particular embodiment, the porous carbon material is prepared by the following method:
a) Heating a reaction mixture comprising:
   an aqueous solution consisting of water and absolute ethanol with a mass water fraction from 1 to 0.5, and,
   xylose and catechin (molar ratio 2:1) dissolved in the aqueous solution, at a temperature below 300° C. (preferably 180° C.) to obtain a solid porous carbon material,
b) Washing the solid porous carbon-based material obtained by successive soaking in a solvent, preferably a polar solvent.

According to a preferred embodiment, the porous carbon material consists of
40-70% by weight in carbon element (C)
30-40% by weight in oxygen (O) element
preferably 60% by weight in the element carbon (C) and 35% by weight in the element oxygen (O).

According to a preferred embodiment, the porous carbon material consists of 36% 0 and 60% C for the xylose/catechin system or catechu extract.

According to a preferred embodiment, the porous carbon-based material is formed from at least one carbohydrate (c1), preferably at least one ose, or at least one polysaccharide (c1) and at least one compound having at least an orthodiphenol or orthomethoxyphenol function (c2) or in a molar ratio (c1/c2) from 10:1 to 1:10, preferably 2/1.

The solid porous carbon material is washed by soaking either in ultrapure water, deionized water or a polar protic water/solvent mixture, e.g. methanol, ethanol or tert-butanol for several hours.

The purpose of washing is to extract soluble compounds not incorporated into the structure of the porous carbon-based material and to prepare the gel for the drying stage.

The carbon-based material obtained or capable of being obtained in the hydrothermal carbonization step (a) is typically composed of primary nanoparticles aggregated into an interconnected network forming the dispersed phase, with the aqueous solution forming the dispersion phase.

The carbon-based material before drying can be called solvogel. When the aqueous solution is water, the carbon-based material obtained in step (a) can be called a hydrogel.

Therefore, a hydrogel is considered here as a special type of solvogel, in which the aqueous solution of the dispersion phase is water.

The solid porous carbon material obtained or capable of being obtained by the method described above may be dried and transformed into cryogel, aerogel or xerogel.

According to a first variant, a dry form of a hydrogel or solvogel is obtained by subjecting the carbon-based material (i.e. hydrogel or solvogel) to contact with a supercritical fluid, in particular acetone or carbon dioxide, to transform it into an aerogel.

According to a second variant, a dry form of hydrogel is obtained by subjecting the carbon-based material (i.e. hydrogel or solvogel) to freezing the isolated gel and freeze-drying the frozen gel to transform it into a cryogel.

According to a third variant, a dry form of hydrogel is obtained by subjecting the carbon-based material (i.e. hydrogel or solvogel) to an evaporation of the solvent at controlled temperature and pressure to transform it into a xerogel.

Alternatively, a cryogel can be called an aerogel when the dried gel has largely retained the textural properties after drying.

Dried gels can be called aerogels regardless of the drying technique used.

Depending on the gel drying method, it is possible to control the pore size and structure of the porous carbon materials obtained according to the invention.

In a particular embodiment, the porous carbon material is prepared by the following method:
a) Heating a reaction mixture comprising an aqueous solution, and at least two bio-based precursors #1 and 2 as defined above dissolved in the aqueous solution, at a temperature below 300° C., preferably at 180° C. to obtain a solid porous carbon-based material,
b) Washing the solid porous carbon material obtained by successive soaking in a polar solvent (ideally in absolute ethanol or in a water/tert-butanol mixture 75/25 in mass percentages),
c) Drying the solid porous carbon-based material using a supercritical fluid or by freeze-drying.

The hydrogels placed in borosilicate glass beakers are previously immersed in liquid nitrogen (77 K) for 15 minutes and then placed directly in the freeze dryer chamber (e.g. a COSMOS freeze dryer from Cryotec). Drying is carried out under vacuum <50 mTorr for 48 hours with a cold trap at −80° C. which ensures the condensation of water vapours or solvents.

Supercritical drying and freeze-drying are preferred to maintain the pore system. According to a particularly preferred embodiment, the solvent is removed by extraction with supercritical CO₂ for this method, the solvent in the solvogel is preferably chosen from absolute ethanol or acetone (e.g. supplied by solvent replacement by absolute ethanol or acetone).

### Porous Carbon Material Texture

The porous carbon-based material obtained or which can be obtained in step (a), is a gelled material, in particular a solidified material composed of aggregated particles forming an interconnected network.

The porous carbon-based material obtained or capable of being obtained by the method described above comprises micropores (pores <2 nm), mesopores (pores of 2-50 nm) and macropores (pores >50 nm).

In addition, in contrast to conventional activated carbons (e.g. those produced by pyrolysis), the oxygen content is typically greater than 25% for materials directly obtained in the hydrothermal carbonization step (a).

The surface of the carbon-based material of the invention as obtained in step (a) is very hydrophilic.

### Textural Properties of Porous Carbon Material

### Pore Diameter

The porous carbon material according to the present invention is composed of aggregated particles forming an interconnected network. This interconnected network of aggregated particles induces interparticle interstices (or pores) with a diameter from 10 μm and 5 nm, preferably with an average diameter from 5 to 200 nm, more preferably with an average diameter from 25 to 100 nm and even more preferable from 40 to 80 nm and preferably around 60 nm.

In addition, the porous carbon material according to the present invention is an aggregate matter composed of particles, with a pore size distribution calculated from a nitrogen adsorption/desorption isotherm at 77 K (for the narrowest pores; <100 nm) and a mercury intrusion porosimetry profile (for the widest pores; >50 nm).

In addition, the pore size distribution and the pore diameter that corresponds to the peak of the pore size distribution can be provided by BJH analysis of the nitrogen adsorption/desorption isotherm.

The pore diameter of the carbon gel according to the present invention, corresponding to the peak of the pore size distribution, is preferably in the range from 40 to 80 nm.

It should be noted that, in order to prevent the reduction of the amount of adsorbed components, the pore diameter of the porous carbon-based material according to the present invention, corresponding to the peak of the pore size distribution, is preferably greater than the average molecular diameter of the diameter of the adsorbed or complexed components.

### Specific Surface

The porous carbon-based material according to the present invention preferably has a specific surface area from 300 to 1000 m²/g, more preferably has a specific surface area from 600 to 800 m²/g.

If the porous carbon-based material has a specific surface area of less than 100 m²/g, the surface area with which the adsorbed components are in contact will be reduced, and the number of pores in which the adsorbed components are housed will be reduced.

### Porous Volume

In addition, the total pore volume of the porous carbon-based material according to the present invention is not particularly limited because it also varies with the specific surface area and pore diameter which corresponds to the peak of the pore size distribution. However, the porous carbon-based material according to the present invention preferably has a total pore volume from 0.1 to 5 cm³/g, more preferably has a total pore volume from 0.2 to 2.5 cm³/g.

The so-called total volume was determined at a relative pressure of 0.99 on the nitrogen adsorption/desorption isotherms at −196° C. The surface area and volume of micropore were determined by applying the t-plot method. The pore diameter distributions were evaluated by applying the BJH method on the isothermal desorption branch. Aerogels were degassed for at least 6 h at 0.05 mbar on a VacPrep Micromeritics before analysis. The analyses were performed on a TriStar Micromeritics. The total pore volume in the materials is greater than that obtained by nitrogen sorption volume at −196° C., since the largest pores (>100 nm in diameter) cannot be analyzed by this method. The largest pores (>100 nm in diameter) were analyzed by mercury intrusion porosimetry. The volume of these large pores is greater than 3 mL/g for catechin/xylose aerogels.

The specific surface area and total porous volume of the porous carbon material according to the present invention can be determined by a general volumetric measurement described below. Specifically, a porous carbon-based material is placed in a container and cooled to the temperature of liquid nitrogen (−196° C.). Then, nitrogen gas is introduced into the container and the amount of nitrogen gas adsorbed on the carbon gel is determined based on the volumetric method. Then, the nitrogen gas pressure introduced into the container is gradually changed and the amount of nitrogen gas adsorbed onto the carbon gel is plotted according to each equilibrium pressure. This results in the nitrogen adsorption/desorption isotherm.

In addition, the specific surface area can be determined by the Brunauer Emmett Teller method (BET), e.g. by the nitrogen adsorption technique.

The specific surface area equivalent to BET was determined by nitrogen adsorption at 77K by making isotherms at relative pressures P/P0 from 0.01 and 0.99. The BET method was applied over a range of relative pressures from 0.1 and 0.3 (based on the Rouquerol transform).

According to the present invention, an aerogel prepared from catechin mixtures or a natural extract of catechu and an ose (see FIGS. 3 and 4) has a total volume from 0.5 to 0.8 cm³/g, an external volume from 0.5 to 0.8 cm³/g, and a microporous volume from 0.01 to 0.05 cm³/g.

### Interconnected Porosity

This parameter can be determined by mercury intrusion porosimetry indicating the total volume of interconnected pores.

### Ion Complexation

The porous carbon-based materials as described above are complexed with metal ions, preferably at least one type of metal cation which is complexed on the outer surfaces and the inner pore surfaces of the porous carbon-based material according to the invention.

According to a preferred mode of the invention, the at least one metal cation is selected from Al³⁺, Ag⁺, Ba²⁺, Ca²⁺, Cd²⁺, Co²⁺, Cr³⁺, Cu⁺, Cu²⁺, Fe²⁺, Fe³⁺, K⁺, Mg²⁺, Mn²⁺, Na⁺, Ni²⁺, Pb²⁺, Sn²⁺, Sn⁴⁺, Zn²⁺, preferably one or more cations of transition metals Fe³⁺, Co²⁺, Zn²⁺, Cu²⁺, or Ni²⁺.

The metal cation is complexed or chelated with porous carbon-based material. A chelate is a type of coordination compound in which a single metal ion is attached by coordinated covalent bonds to a molecule or ion called a ligand. A coordination compound is formed when groups of atoms, ions, or molecules chemically bond to each other by donating and accepting pairs of electrons. The groups that give electron pairs are called ligands. These are usually Lewis bases. Groups accepting electron pairs are often transition metal cations.

According to the present invention, the term “hybrid material” refers to the porous carbon-based material on which the metal ions are complexed. This is the composition within the meaning of the present invention.

In a particular embodiment, the composition (porous carbon-based hybrid material) is prepared by the following method:
a) Heating a reaction mixture comprising an aqueous solution, and, at least two bio-based precursors #1 and 2 as defined above dissolved in the aqueous solution, to a temperature below 300° C. to obtain a solid porous carbon-based material,
b) Washing the solid porous carbon material obtained by successive soaking in a polar solvent,
c) Drying the solid porous carbon-based material using a supercritical fluid or by freeze-drying,
d) Impregnate the solid porous carbon material with a solution comprising metal cations to obtain a porous carbon hybrid material complexed with metal cations.

The adsorption method to allow the metal complex to adsorb to a carbon-based material, the adsorption conditions are not particularly limited.

For example, a metal complex can be adsorbed onto a carbon-based material by placing the porous carbon-based material in a solution and mixing the solution for a period of time at from about 10 to 100° C. The hybrid material is separated from the solution by centrifugation, and the resulting suspension is washed and dried.

According to another variant of the invention, the composition (porous carbon-based hybrid material) is prepared by the following method:
a) Heating a reaction mixture comprising an aqueous solution, and, at least two bio-based precursors #1 and 2 as defined above dissolved in the aqueous solution, with a solution comprising metal cations, to a temperature below 300° C. to obtain a solid porous carbon-based material,
b) Washing the solid porous carbon hybrid material obtained by successive soaking in a polar solvent,
c) Drying the solid porous carbon hybrid material using a supercritical fluid or by freeze-drying.

The amount of metal ions complexed with the porous carbon-based material to form the hybrid material or composition of the present invention is not particularly limited. However, the amount of metal ions adsorbed or complexed on a carbon-based material is preferably from 0.1 and 40 parts by weight compared to 100 parts by weight of the porous carbon-based material.

In a particular embodiment, the composition comprises a quantity of chelated or complexed metal cations, measured by the SEM-EDX method, ranging from 0.2 to 2% molar, preferably from 0.5 to 1% molar.

In a particular embodiment, the composition comprises a quantity from 1 to 5% by mass of metal cation, in particular from 2 to 4% by mass.

In a particular embodiment, the composition comprises a quantity from 20 to 40 mg of iron per gram of porous carbon-based material.

The solvent used here is not particularly limited as long as it can dissolve and/or disperse metal complexes. Examples of these include: acetic acid, water, ethylene glycol, DMSO, and DMF.

The concentration of metal complex in the solution is not particularly limited. However, the concentration is preferably from about 0.1 to 500 mM.

According to a preferred mode of the invention, the composition (solid porous carbon hybrid material) comprises:
(a) a porous carbon-based material obtained by hydrothermal carbonization comprising or formed from:
   at least one carbohydrate, preferably at least one ose, or at least one polysaccharide (bio-based precursor #1), and
   at least one compound with at least one orthodiphenol or orthomethoxyphenol function (bio-based precursor #2), and
(b) at least one metal cation selected from Al³⁺, Ag⁺, Ba²⁺, Ca²⁺, Cd²⁺, Co²⁺, Cr³⁺, Cu⁺, Cu²⁺, Fe²⁺, Fe³⁺, K⁺, Mg²⁺, Mn²⁺, Na⁺, Ni²⁺, Pb²⁺, Sn²⁺, Sn⁴⁺, Zn²⁺, preferably one or more cations of transition metals Fe³⁺, Co²⁺, Zn²⁺, Cu²⁺, or Ni²⁺.

According to a preferred mode of invention, the composition comprises:
(a) a porous carbon-based material obtained by hydrothermal carbonization comprising or formed from:
   of at least one carbohydrate, preferably at least one ose, or at least one polyoside, in which
      The ose is chosen from among
         an aldose chosen from: glyceraldehyde, erythrose, threose, ribose, arabinose, xylose, lyxose, allose, altrose, glucose, mannose, gulose, idose, galactose, and talose, preferably xylose,
      or
         a ketose chosen from: dihydroxyacetone, erythrulose, ribulose, xylulose, fructose, psicose, sorbose, and tagatose, preferably fructose, or
      the polysaccharide is chosen from:
         a heterodiholoside chosen from: trehalulose, sucrose, turanose, maltulose, leucrose, isomaltulose, gentiobiulose, melidiose, lactulose, lactose, and rutinose, or
         a homodiholoside chosen from: inulobiose, alpha2-mannobiose, alpha3-mannobiose, trehalose, kojibiose, nigerose, maltose, isomaltose, sophorose, laminaribiose, cellobiose, and gentiobiose, preferably maltose or sucrose
and
   at least one compound with at least one orthodiphenol or orthomethoxyphenol function (bio-based precursor #2), and
(b) at least one metal cation selected from Al³⁺, Ag⁺, Ba²⁺, Ca²⁺, Cd²⁺, Co²⁺, Cr³⁺, Cu⁺, Cu²⁺, Fe²⁺, Fe³⁺, K⁺, Mg²⁺, Mn²⁺, Na⁺, Ni²⁺, Pb²⁺, Sn²⁺, Sn⁴⁺, Zn²⁺, preferably one or more cations of transition metals Fe³⁺, Co²⁺, Zn²⁺, Cu²⁺, or Ni²⁺.

In another preferred mode of invention, the composition comprises:
(a) a porous carbon-based material obtained by hydrothermal carbonization comprising or formed from:
   of at least one carbohydrate, preferably at least one ose, or at least one polyoside, in which
   The ose is chosen from among
      an aldose chosen from: glyceraldehyde, erythrose, threose, ribose, arabinose, xylose, lyxose, allose, altrose, glucose, mannose, gulose, idose, galactose, talose, preferably xylose,
   or
      a ketose chosen from: dihydroxyacetone, erythrulose, ribulose, xylulose, fructose, psicose, sorbose, tagatose, preferably fructose, or
   the polysaccharide is chosen from:
      a heterodiholoside chosen from: trehalulose, sucrose, turanose, maltulose, leucrose, isomaltulose, gentiobiulose, melibiose, lactulose, lactose, rutinose, or
      a homodiholoside chosen from: inulobiose, alpha2-mannobiose, alpha3-mannobiose, trehalose, kojibiose, nigerose, maltose, isomaltose, sophorose, laminaribiose, cellobiose, gentiobiose, preferably maltose or sucrose,
      at least one compound with an orthodiphenol or orthomethoxyphenol function chosen from: catechol, pyrogallol, caffeic acid, mangaferine, quercetin, cyanidin, catechin, epicatechin, epigallocatechin, anthocyanidol, procyanidol B-3, procyanidol B-4, fuhalols (bifuhahol, trifuhahol), carmalol (diphlorethohydroxycamalol) and tannic acid or a natural plant extract containing at least one of the above compounds
and
(b) at least one metal cation selected from Al³⁺, Ag⁺, Ba²⁺, Ca²⁺, Cd²⁺, Co²⁺, Cr³⁺, Cu⁺, Cu²⁺, Fe²⁺, Fe³⁺, K⁺, Mg²⁺, Mn²⁺, Na⁺, Ni²⁺, Pb²⁺, Sn²⁺, Sn⁴⁺, Zn²⁺, preferably one or more cations of transition metals Fe³⁺, Co²⁺, Zn²⁺, Cu²⁺, or Ni²⁺.

In another preferred mode of invention, the composition comprises:
(a) a porous carbon-based material obtained by hydrothermal carbonization comprising or formed from:
   xylose, or dihydroxyacetone,
   catechin, and
b) at least one metal cation selected from Fe³⁺, Co²⁺, Zn²⁺, Cu²⁺, or Ni²⁺.

In another preferred mode of invention, the composition comprises:
(a) a porous carbon-based material obtained by hydrothermal carbonization comprising or formed from:
   xylose,
   catechin, and
b) at least one metal cation that is Fe³⁺

In another preferred mode of invention, the composition comprises:
(a) a porous carbon-based material obtained by hydrothermal carbonization comprising or formed from:
   xylose,
   catechin, and
b) at least one metal cation that is Co²⁺

In another preferred mode of invention, the composition comprises:
(a) a porous carbon-based material obtained by hydrothermal carbonization comprising or formed from:
   dihydroxyacetone,
   catechin, and
b) at least one metal cation that is Fe³⁺

According to the present invention, all the compositions described above can be used to immobilize proteins, in particular at least one enzyme

### Immobilization of a Protein

A second purpose of the invention is to propose a composition comprising a protein or enzyme immobilized on said solid porous carbon hybrid material or composition. The solid porous carbon hybrid material of the present invention comprises the solid porous carbon material described above as a support, at least one metal ion complexed to said material and at least one protein (in particular an enzyme) immobilized (on the porous carbon material loaded with at least one metal cation), in which the at least one immobilized protein preferably contains an affinity tag and is immobilized by so-called weak bonds (bonds van der Waals, hydrogen bonds) and/or coordination bonds and/or ionic bonds.

The protein used here is not particularly limited.

The composition of the present invention thus comprises at least one enzyme selected from the approved list of the nomenclature and classification of enzymes of the International Union of Biochemistry (IUBMB) and examples thereof comprise the following proteins:
EC 1 Oxidation Reductases, EC 2 Transferases, EC 3 Hydrolases, EC 4 Lyases, EC 5 Isomerases, EC 6 Ligases and EC 7 Translocases.

Among these proteins, at least one protein selected from the group consisting of, in particular, α-arabinosidase, α-galactosidase, α-rhamnosidase, β-galactosidase, β-glucanase, β-glucosidase, β-glucanase, β-mannanase, γ-lactamase, acetolactate decarboxylase, activase, adenosine deaminase, aminoacylase, aminopeptidase, amylase, amyloglucosidase, asparginase, aspartase, bromelain, carbonic anhydrase, catalase, cellulase, chitinase, chymosin, collagenase, cyclodextrinase, deoxyribonuclease I, dextranase, enoate reductase, epimerase, esterase, formate dehydrogenase, galactinol synthase, glucanotransferase, glucoamylase, glucose isomerase, glucose oxidase, glutenase, hemicellulase, hexose oxidase, inulinase, invertase, laccase, lactase, lactate dehydrogenase, leucine dehydrogenase, levanase, lipase, lipoxygenase, lysozyme, methane monooxygenase, monoamine oxidase, muramidase, naphthalene dioxygenase, naphthalene monooxygenase, naringinase, nattokinase, nitrile hydratase, papain, pectinase, pectinesterase, penicillin G, acylase, pentosanase, phenoloxidase, phenylalanine dehydrogenase, phytase, polyethylesterase, polygalacturonase, protease, protopectinase, pullulanase, pyrophosphatase, pyruvate transaminase, raffinose synthase, raffinose synthase, rennet, sacrosidase, serratiopeptidase, sphingosine kinase, stachyose synthase, tannase, taxolase, thermolysine, transaminase, transglutimases, trypsin, urease, xylanase, xylose isomerase.

According to another preferred mode of the invention, the composition comprises a protein selected from the group consisting in particular of an enoate reductase (EC 1.3.1.31), a transaminase (EC 2.6.1), a pyrophosphatase (EC3.6.1.1).

### Protein Loads

With respect to the porous carbon-based hybrid material of the present invention, the amount of protein adsorbed on the porous carbon-based material is not particularly limited as long as it exhibits enzymatic activity. However, the amount of protein adsorbed on the porous carbon-based material is preferably from 10 to 80 parts by weight compared to 100 parts by weight of the porous carbon-based material, preferably from 10 to 50 parts by weight compared to 100 parts by weight of the porous carbon-based material.

In addition, the method for providing the porous carbon-based hybrid material of the present invention by causing the adsorption of a protein on porous carbon-based material is not particularly limited. It is possible to use, for example, the impregnation method. The impregnation method described below is more preferable. That is, the protein is first dissolved in water or buffer at a concentration at which the protein is not precipitated (preferably at a concentration from 0.1 to 1,000 mg/ml). A porous carbon-based material is then suspended in the resulting solution at a temperature at which the solution does not freeze and at which the protein does not denature (preferably from 0 to 50° C.). Thus, the protein is brought into contact with the porous carbon material for at least 5 minutes or more, preferably for 30 minutes or more and thus the protein is immobilized in the pores of the carbon gel. In this way, the carbon-based hybrid material of the present invention can be supplied.

In a particular embodiment, the composition comprising at least one immobilized protein is prepared by the method comprising the steps of:
Suspend the dried porous carbon material in a solution containing metal cations, to obtain a porous carbon hybrid material loaded with metal cations
Washing porous carbon hybrid material loaded with metal cations
Immobilize a protein (enzyme) on the porous carbon hybrid material loaded with metal cations by reacting the porous carbon-based material loaded with metal cations with said protein preferably bearing an affinity marker, preferably a poly-histidine tag;
Washing the suspension and recover the protein immobilized on the material porous carbon-based hybrid loaded with metal cations.

More specifically, the porous carbon-based hybrid material loaded with metal cations is added to an aqueous solution containing a protein preferably labelled with a poly-histidine label at its N-terminal or C-terminal.

As used here, the term “affinity-labelled protein” refers to a recombinant protein in which an affinity marker, as defined above, has been added to the target protein.

Affinity-labeled proteins can be prepared by recombinant DNA technology using methods known in the technique, such as DNA fragment ligation or by PCR techniques. Affinity-labeled proteins can also be referred to as “fusion-labeled proteins” or “fusion proteins.”

The term “polyhistidine tag” refers to a chain of two or more histidine residues, which is attached to the C or N end of a protein.

The polyhistidine marker is preferably a chain of at least six histidine residues.

The term “polyhistidine-labeled enzyme” refers to a recombinant enzyme in which the target enzyme is fused with a polyhistidine tag as defined above.

The term “affinity marker” refers to a defined group, such as an organic or organometallic molecule, a protein fragment, or other, that is attached to a recombinant protein and is able to bind to a specific group immobilized on a matrix.

The porous carbon-based hybrid material loaded with metal cations on which at least one protein is immobilized by so-called weak bonds (van der Waals bonds, hydrogen bonds) and/or coordination bonds and/or ionic bonds, is washed several times with a buffered aqueous solution.

The affinity marker used in the invention may be any marker capable of binding specifically to the porous carbon-based material for which it has an affinity.

Affinity bonding can be the result, for example, of the van der Waals interaction, hydrogen bonding, ion bonding, or hydrophobic interaction.

In all cases, the affinity bond must be strong enough to allow the affinity marker and the porous carbon material to remain tightly bound to each other at least until certain specific conditions are applied in order to dissociate the affinity marker from the porous carbon material.

The protein to be immobilized on the material can be any protein containing an affinity marker, such as a (recombinant) protein or an enzyme containing an affinity marker.

Preferably, the protein is an enzyme containing an affinity marker.

A number of affinity labels and corresponding matrices are known in the technique.

In a preferred embodiment, the affinity marker on the protein is a polyhistidine marker and the solid material contains a chelated metal ion.

The buffered aqueous solution is preferably Tris-HCl, or Phosphate buffer.

According to the present invention, an affinity marker is a marker which has a specific affinity for metal cations.

According to a second aspect of the invention, the present application relates to a composition comprising:
(a) a porous carbon-based material formed from:
   at least one carbohydrate, preferably at least one ose, or at least one polysaccharide (bio-based precursor #1), and
   at least one compound with an orthodiphenol or orthomethoxyphenol function (bio-based precursor #2),
(b) at least one metal cation, and
(c) at least one protein of preference comprising a poly-histidine tag

According to a preferred mode of invention, the composition comprises
(a) a porous carbon-based material formed from:
   at least one carbohydrate, preferably at least one ose, or at least one polysaccharide (bio-based precursor #1), and
   at least one compound with an orthodiphenol or orthomethoxyphenol function (bio-based precursor #2),
(b) at least one metal cation, and
(c) at least one immobilized protein, preferably at least one immobilized protein comprising a poly-histidine tag.

In another preferred mode of invention, the composition comprises:
(a) a porous carbon-based material formed from:
   of at least one carbohydrate, preferably at least one ose, or at least one polyoside, from which the ose is chosen from:
      an aldose chosen from: glyceraldehyde, erythrose, threose, ribose, arabinose, xylose, lyxose, allose, altrose, glucose, mannose, gulose, idose, galactose, and talose, preferably xylose,
   or
      a ketose chosen from: dihydroxyacetone, erythrulose, ribulose, xylulose, fructose, psicose, sorbose, and tagatose, preferably fructose,
   or from which the polysaccharide is chosen from:
      a heterodiholoside chosen from: trehalulose, sucrose, turanose, maltulose, leucrose, isomaltulose, gentiobiulose, melidiose, lactulose, lactose, and rutinose, or
      a homodiholoside chosen from: inulobiose, alpha2-mannobiose, alpha3-mannobiose, trehalose, kojibiose, nigerose, maltose, isomaltose, sophorose, laminaribiose, cellobiose, and gentiobiose, preferably maltose or sucrose, and
   at least one compound with an orthodiphenol or orthomethoxyphenol function chosen from: catechol, pyrogallol, caffeic acid, mangiferine, quercetin, cyanidin, catechin, epicatechin, epigallocatechin, anthocyanidol, procyanidol B-3, procyanidol B-4, fuhalols (bifuhahol, trifuhahol), carmalol (diphlorethohydroxycamalol) and tannic acid,
      or
      a natural plant extract containing at least one of the above compounds,
(b) at least one metal cation selected from Al³⁺, Ag⁺, Ba²⁺, Ca²⁺, Cd²⁺, Co²⁺, Cr³⁺, Cu⁺, Cu²⁺, Fe²⁺, Fe³⁺, K⁺, Mg²⁺, Mn²⁺, Na⁺, Ni²⁺, Pb²⁺, Sn²⁺, Sn⁴⁺, Zn²⁺, preferably one or more cations of transition metals Fe³⁺, Co²⁺, Zn²⁺, Cu²⁺, or Ni²⁺, and
(c) at least one immobilized protein, preferably at least one immobilized protein comprising a poly-histidine tag.

In another preferred mode of invention, the composition comprises:
(a) a porous carbon-based material formed from:
   xylose, or dihydroxyacetone,
   catechin, and
b) at least one metal cation selected from Fe³⁺, Co²⁺, Zn²⁺, Cu²⁺, or Ni²⁺ and
(c) at least one immobilized protein, preferably at least one immobilized protein comprising a poly-histidine tag.

In another preferred mode of invention, the composition comprises:
(a) a porous carbon-based material formed from:
   xylose,
   catechin, and
b) at least one metal cation which is Fe³⁺, and
(c) at least one immobilized protein, preferably at least one immobilized protein comprising a poly-histidine tag.

In another preferred mode of invention, the composition comprises:
(a) a porous carbon-based material formed from:
   xylose,
   catechin, and
(b) at least one metal cation that is Co²⁺, and
(c) at least one immobilized protein, preferably at least one immobilized protein comprising a poly-histidine tag.

In another preferred mode of invention, the composition comprises:
(a) a porous carbon-based material formed from:
   dihydroxyacetone,
   catechin, and
b) at least one metal cation which is Fe³⁺, and
(c) at least one immobilized protein, preferably at least one immobilized protein comprising a poly-histidine tag.

For example, if the solid material according to the invention is used for the purification and isolation of an affinity-labeled protein, the binding of the enzyme to the support must be reversible. In such cases, it is preferred that the chelated metal ion be Fe³⁺, Ni²⁺ or Co²⁺. These metal ions bind strongly enough to immobilize a polyhistidine-labeled enzyme, but are also able to release the immobilized enzyme when specific conditions are applied, such as treatment with a buffered solution containing imidazole or ethylenediaminetetraacetate (EDTA).

For the use of immobilized enzymes in heterogeneous biocatalysis, a strong binding of the enzyme to the support is desirable.

In such cases, it is preferable that the chelated metal ion be either Co²⁺ or Fe³⁺, and preferably Fe³⁺ of all others, since this results in a particularly strong binding of the polyhistidine marker to the said material. As demonstrated in the examples, leaching of the enzyme or metal ion from a material comprising a protein immobilized in the presence of Fe³⁺ as a chelated metal ion is almost negligible. The absence of leaching allows the immobilized protein material (the biocatalyst) to be used in catalytic quantities. The use of catalytic quantities is particularly important in flow-through reactions.

Another advantage of Fe³⁺ as a chelated metal ion is that this metal is non-toxic.

The high affinity of the polyhistidine marker for metal ions such as Co²⁺ or Fe³⁺ allows the immobilization of polyhistidine-labeled proteins to be performed from crude solutions containing the proteins without the need for extensive purification of the solution prior to the immobilization step. Organic matter that does not contain a polyhistidine marker binds to chelated metal ions only weakly, or not at all, and is easily removed from material containing at least one polyhistidine-labelled protein, by washing with, for example, water or buffered aqueous solutions. Thus, if the polyhistidine-labeled protein is prepared by intracellular overexpression, protein immobilization can be achieved directly from the cell lysate. Alternatively, if the polyhistidine-labeled protein is secreted by the host organism, protein immobilization can be achieved directly from the cell culture supernatant.

Dissociation of the bound protein can be achieved using standard IMAC methods.

For example, the bound protein can be released from the support by lowering the pH or adding a competitive molecule with equal or greater affinity for chelated metal ions compared to the polyhistidine group, for example by applying a buffered solution containing imidazole or ethylenediaminetetraacetate (EDTA).

After dissociation of the purified proteins from the support, the polyhistidine marker can be removed from the proteins, if necessary, by techniques known in the technique, e.g. by cleaving the affinity marker with an appropriate enzyme such as a specific protease, thus obtaining the protein pure and without a marker.

If the proteins immobilized on the support, as described above, are enzymes, they contain an active site capable of catalyzing a chemical reaction.

Thus, the immobilized enzyme material is potentially useful as a biocatalyst in organic synthesis.

Therefore, in another aspect, the invention relates to the use of an immobilized enzyme material as described herein as a heterogeneous biocatalyst, e.g. in synthetic organic transformations.

A preferred mode of the invention relates to the use of the compositions described above as heterogeneous biocatalysts.

The present invention further relates to a method for catalyzing an enzyme-catalyzed reaction, comprising providing a composition comprising a protein or enzyme immobilized on said porous carbon-based material according to the invention, and bringing said composition comprising an immobilized enzyme into contact with at least one substrate on which the enzyme, which is immobilized on said material, is capable of reacting.

Immobilizing enzymes on said material by affinity tag binding, as described here, improves the stability of the enzymes used.

Immobilized enzymes have been found to tolerate both aqueous conditions as well as a range of organic solvents.

This allows the use of compositions comprising a protein or enzyme immobilized on said porous carbon material under reaction conditions in which the free, non-immobilized enzymes would not have been stable.

It is possible that the composition comprising a protein or enzyme immobilized on said porous carbon material could also be used in a wider pH range than free, non-immobilized enzymes would have tolerated.

Since the native activity of the enzyme is conserved, the composition comprising a protein or enzyme immobilized on said porous carbon-based material has a higher enzymatic activity per protein mass than the initial non-immobilized protein material. For example, the turnover number (TON), defined as the number of moles of product formed per mole of protein over a given time, is greater for an enzyme immobilized according to the invention than for a free enzyme.

The enzyme that is immobilized on said material can be any enzyme that is useful as a biocatalyst in synthetic organic transformations, comprising, but not limited to, enzymes reacting as oxidoreductases, transferases, hydrolases, lyases, isomerases, and ligases.

Thus, compositions comprising an enzyme immobilized on said material can be used as heterogeneous biocatalysts in any organic reaction in which the immobilized enzyme is able to specifically catalyze the reaction.

Examples of such biocatalytic reactions comprise, but are not limited to, enzymatic oxidation and reduction reactions, enzymatic hydrolysis reactions, and enzymatic isomerization reactions.

Particularly useful biocatalytic reactions are enantioselective reactions.

Specific examples of biocatalytic reactions comprise the synthesis of chiral amines from prochiral ketones, the synthesis of esters from carboxylic acids and alcohols or from esters and alcohols, the synthesis of hydroxylated molecules, or the synthesis of terpenes or terpenoids.

In one embodiment, two or more different enzymes can be immobilized on the material according to the invention, wherein each of the different enzymes is capable of catalyzing a different reaction.

It may then be possible to use the material containing two or more different immobilized enzymes as a heterogeneous biocatalyst in a multi-step or cascade reaction.

According to another aspect of the invention, the present application relates to a method for catalyzing an enzyme-catalyzed reaction, comprising providing a composition described above, and contacting said composition with at least one substrate on which an enzyme, which is immobilized on said support of the composition, is capable of reacting.

### Immobilization Yield and Speed

Immobilization yields, expressed in mg of immobilized enzyme per mg of enzyme initially present in the solution, range from 90 to 100% for protein loads of 5 to 50% by mass.

The immobilization speeds expressed in minutes to achieve at least an immobilization rate of at least 75% are from 10 and 120 minutes.

According to a preferred mode of the invention, the method of immobilizing a protein, in particular an enzyme on the porous carbon-based material, is characterized in that the yield of the immobilization is from 90% and 100% for protein loads ranging from 5 to 50% by weight. According to a preferred mode of the invention, the method of immobilizing a protein, in particular an enzyme on the porous carbon-based material, is characterized in that the immobilization speed to achieve an immobilization yield of at least 75% is from 10 and 120 minutes.

According to a final aspect of the invention, the present application relates to the use of the composition as a heterogeneous biocatalyst.

### Enzymatic Performance

The enzymatic performance measured is expressed in the number of reactions catalyzed by enzyme (Turnover number, TON) over a given time. The TON represents the number of moles of substrate converted (or product formed) by the number of moles of enzyme immobilized in a given volume.

According to a preferred mode of the invention, the specific activity (which is the quantity of substrate transformed per unit time and per mass of enzyme) of an enzyme immobilized on the porous carbon-based material is greater than that of the same free enzyme, i.e., not immobilized on said porous carbon-based material.

In Example 3, measurements of the enzymatic performance of several immobilized enzymes show a significantly higher TON than that of the non-immobilized enzyme.

These results are better than those described to date in the literature where it is observed that in most cases the activity of the enzyme is greatly reduced after immobilization.

### EXPERIMENTAL PART

### Example 1: Preparation of a Porous Carbon-Based Material in the Form of an Aerogel (Based on Xylose/Catechin/Fe³⁺ (FeXCat))

0.85 g of xylose is dissolved in 10 mL of a mixture of deionized water and 50/50 m/m absolute ethanol.

0.35 g of catechin is added to the previous solution.
The mixture is stirred until a homogeneous solution is obtained. Ultrasonic treatment at room temperature for one hour or less can accelerate the homogenization of the solution,
The resulting solution is treated by hydrothermal carbonization, i.e. introduced into a mineralization bomb (autoclave), then it is hermetically sealed and placed in a thermostatic chamber at 180° C. for 20 hours,
At the end of this period, the autoclave and its contents are cooled to room temperature for a few hours (usually 2 hours),
The autoclave is then opened and its contents poured into either ultrapure water (usually 100 mL), deionized water, a polar protic water/solvent mixture (methanol, ethanol, propanol, butanol or their isomers), or a pure polar protic solvent.
The resulting solid is washed by successive soaking (usually 3 times 100 mL) with either in ultrapure water, or in deionized water, or in a polar protic water/solvent mixture (methanol, ethanol, propanol, butanol or their isomers), or in a pure polar protic solvent for several hours (usually 8 hours). This results in a hydrogel. The hydrogel is then dried by freeze-drying or in a supercritical fluid to obtain an aerogel.

Aerogels obtained under the conditions described above (200 mg) are added to 10 mL of an aqueous solution containing 400 mmol/L of FeCl₃

The suspension thus obtained is stirred for 4 hours at 20 rpm at room temperature.

After agitation, the suspension is centrifuged (5 min at 5000 rpm). After centrifugation, the supernatant is removed.

The solid thus obtained is washed with water 3 times:
1) Water is added (10 mL),
2) The suspension thus obtained is stirred for a few minutes (usually 5 minutes) at room temperature,
3) The suspension is centrifuged (5 min at 5000 rpm),
4) After centrifugation, the supernatant is removed.

Steps 1 to 4 are repeated twice.

### Example 2: Immobilization of a Protein

10 mg Fe³⁺ ion-complexed aerogel under the conditions described above is added to an aqueous solution containing (1 mg) protein with a polyhistidine tag at its N-terminal end.

The following three enzymes were prepared: Trypanosoma brucei pyrophosphatase (TbVSP1, EC3 Hydrolase), transaminase B9L0N2 (w-TA, EC2 transferase) and Galdieria sulphuraria old Yellow Enzyme (GsOYE, EC1 Oxidoreductase).

The suspension thus obtained is agitated at 750 rpm for a few hours (usually 2 hours) at 4° C.

After stirring, the suspension is centrifuged (from 4000 to 6000 rpm for 2 to 5 min). After centrifugation, the supernatant is removed. The solid thus obtained is washed 2 times with an aqueous solution buffered:
1) Buffer is added (1 mL),
2) The suspension thus obtained is stirred for a few minutes (usually 5 minutes) at room temperature,
3) The suspension is centrifuged (from 4000 to 6000 rpm for 2 to 5 min),
4) After centrifugation, the supernatant is removed.

Steps 1 to 4 are repeated 1 time.

This results in a protein carrying a polyhistidine sequence immobilized on porous carbon material complexed with Fe³⁺ ions.

### Variable Protein Loads

Aerogels obtained under the conditions described above (10 mg) are added to a buffer solution (Tris HCl 50 mM NaCl 150 mM pH=7.5, 1 mL) containing 1/2.5/5 mg of GsOYE enzyme depending on the desired protein load (10/25/50% by weight) or 1/2.5 mg of enzyme B9L0N2 for a protein load of 10 and 25% by weight.

The suspension thus obtained is agitated at 800 rpm for 2 hours at 4° C.

For the kinetic monitoring of immobilization, samples are taken at 10, 20, 30, 60, 90 and 120 min. To do this, the suspension is centrifuged from 4000 to 6000 rpm for 2 to 5 min and then a 20 μL sample is taken. The stirring of the reaction mixture is resumed immediately after the sampling is taken.

After the last sampling, the entire supernatant is removed.

The solid thus obtained is washed twice with a buffer solution:

1) 1 mL of buffer is added.

2) The suspension thus obtained is stirred for a few minutes (usually 5 minutes) at room temperature.

3) The suspension is centrifuged (from 4000 to 6000 rpm for 2 to 5 min).

4) After centrifugation, the supernatant is removed. Steps 1 to 4 are repeated once.

The amount of protein present in the samples is measured by Bradford assay.

This results in the GsOYE protein immobilized on a hydrogel (aerogel) with a protein load varying from 10 to 50% by mass and the B9L0N2 protein immobilized on a hydrogel (aerogel) with a protein load varying from 10% to 25% by mass.

### Continuous Flow Immobilization:

### B9L0N2

The FeXCat aerogels obtained under the conditions described above (50 mg) are introduced into a non-thermostatic column whose end is made up of a sinter to retain the aerogel.

A buffer solution (Phosphate 50 mM NaCl 300 mM pH=7.5, 5 mL) containing 5 mg of the enzyme B9L0N2 percolates at a rate of 0.1 mL/min into the column through a pump. The solution is retrieved at the exit of the column. The aerogel contained in the column is then washed by percolation of buffer solution at a rate of 0.1 mL/min for 50 min. The solution is also retrieved at the exit of the column.

The amount of protein present in these two solutions is measured by Bradford assay.

The result is the B9L0N2 protein immobilized on a FeXCat hydrogel (with a protein load of 10% by mass) in a column for use in continuous flow and recirculation.

### GsOYE

The FeXCat aerogels obtained under the conditions described above (50 mg) are introduced into a non-thermostatic column whose end is made up of a sinter to retain the aerogel.

A buffer solution (Tris HCl 50 mM NaCl 150 mM pH=7.5, 5 mL) containing 5 mg of the enzyme GsOYE4 percolates at a rate of 0.1 mL/min into the column through a pump. The solution is retrieved at the exit of the column. The aerogel contained in the column is then washed by percolation of buffer solution at a rate of 0.1 mL/min for 50 min. The solution is also retrieved at the exit of the column.

The amount of protein present in these two solutions is measured by Bradford assay.

The GsOYE protein is thus immobilized on a FeXCat hydrogel (with a protein load of 10% by mass) in a column for use in recirculation.

### Example 3 Immobilization Kinetics

10 mg porous carbon-based material complexed with Fe³⁺ ions is presented with 1 mg of enzyme labelled polyhistidine in a total volume of 1 mL.

Immobilization of transaminase B9L0N2 occurs in the presence of the pyridoxal phosphate cofactor (PLP).

Immobilization of TbVSP1 pyrophosphatase occurs in the presence of the Mg2+ cofactor.

Samples 0, 10, 20 and 30 min (10 μL) are diluted to half and 1 h and 2 h samples (20 μL) are not diluted.

20 μL sample (Ech1 to 4) mixed with 1 mL of Bradford reagent, in the dark for 15 min.

### Results

TABLE 2
Kinetics of TbVSP1 immobilization (1 mg) on a Xylose/Catechin
aerogel complexed with Fe³⁺ ions (10 mg) prepared
under the conditions described above.

| Time | Sample 1 | Sample 2 | Sample 3 | | Standard |
|---|---|---|---|---|---|
| (min) | Immobilized rate/free enzyme (%) | | | Average | deviation |
| 0 | 0 | 0 | 0 | 0 | 0 |
| 10 | 72 | 43 | 39 | 51 | 14 |
| 20 | 92 | 100 | 97 | 96 | 3 |
| 30 | 100 | 100 | 100 | 100 | 0 |
| 60 | 100 | 100 | 98 | 99 | 1 |
| 120 | 100 | 100 | 100 | 100 | 0 |

TABLE 3
Kinetics of B9L0N2 immobilization (1 mg) on a Xylose/Catechin
aerogel complexed with Fe³⁺ ions (10 mg) prepared
under the conditions described above.

| Time | Sample 1 | Sample 2 | Sample 3 | Sample 4 | Aver- | Standard |
|---|---|---|---|---|---|---|
| (min) | Immobilized rate/free enzyme (%) | | | | age | deviation |
| 0 | 0 | 0 | 0 | 0 | 0 | 0 |
| 10 | 67 | 73 | 77 | 53 | 68 | 7 |
| 20 | 65 | 70 | 88 | 70 | 73 | 8 |
| 30 | 84 | 72 | 96 | 68 | 80 | 10 |
| 60 | 97 | 82 | 95 | 85 | 90 | 6 |
| 120 | 96 | 86 | 98 | 88 | 92 | 5 |

TABLE 4
Kinetics of immobilization of GsOYE (1 mg) on a Xylose/Catechin
aerogel complexed with Fe³⁺ ions (10 mg) prepared
under the conditions described above.

| Time | Sample 1 | Sample 2 | Sample 3 | | Standard |
|---|---|---|---|---|---|
| (min) | Immobilized rate/free enzyme (%) | | | Average | deviation |
| 0 | 0 | 0 | 0 | 0 | 0 |
| 10 | 94 | 85 | 96 | 92 | 4 |
| 20 | 98 | 97 | 97 | 97 | 0 |
| 30 | 98 | 98 | 98 | 98 | 0 |
| 60 | 98 | 98 | 98 | 98 | 0 |
| 120 | 98 | 98 | 98 | 98 | 0 |

The results of Tables 2-4 show that the immobilization rates to achieve an immobilization rate of at least 75% are from 10 and 120 minutes.

These results show that the immobilization of an enzyme on porous carbon-based material complexed with Fe³⁺ ions is simple, fast and efficient.

### Example 4: Performance

Conversion rates are expressed in moles of product formed per moles of substrate initially present, measured and calculated after a given reaction time.

For TbVSP1, the reaction studied is the hydrolysis of pyrophosphate to phosphate in the presence of the cofactor Mg²⁺. The protein load used is 10% by mass. The reaction mixture consists of sodium pyrophosphate (5 mM), MgCl (1 mM) in solution in Tris-HCl buffer 50 mM NaCl 200 mM pH 8.5. The solid consisting of 1 mg of TbVSP1 carrying a polyhistidine sequence immobilized on a hydrogel complexed with Fe³⁺ ions (10 mg) is added to the reaction mixture. Phosphate formation is monitored by determination of phosphmolybdate/malachite green in visible spectroscopy at 630 nm. The results are summarized in Table 5.

For w-TA, the reaction studied is the transamination of pyruvate in the presence of racemic α-methylbenzylamine to alanine and acetophenone in the presence of the pyridoxal phosphate cofactor (PLP). The protein load used is 10% by mass. The reaction mixture consists of pyruvate (25 mM), racemic α-methylbenzylamine (25 mM), PLP (0.1 mM) and DMSO (1% wt) in solution in 50 mM NaCl Phosphate buffer, 300 mM pH 7.5. The solid consisting of 1 mg of w-TA carrying a polyhistidine sequence immobilized on a hydrogel complexed with Fe³⁺ ions (10 mg) is added to the reaction mixture. The formation of acetophenone is continuously monitored by UV spectroscopy at 245 nm. The results are summarized in Table 5.

For GsOYE, the reaction studied is the reduction of cyclohexenone and cyclohexanone in the presence of the cofactor NADH which oxidizes to NAD+. The protein load used is 10% by mass. The reaction mixture consists of cyclohexenone (50 mM), NADH (75 mM) and DMSO (1% wt) in solution in Tris-HCl buffer 50 mM NaCl 150 mM pH 7.5. The solid consisting of 1 mg of GsOYE carrying a polyhistidine sequence immobilized on a hydrogel complexed with Fe³⁺ ions (10 mg) is added to the reaction mixture. The disappearance of NADH is monitored by UV spectroscopy at 340 nm. The results are summarized in Table 5.

TABLE 5
Catalytic performance (turnover numbers, TON and Conversion rate)
of the 3 free or immobilized enzymes in the presence of Fe³⁺ ions

| | w-ta | TbVSP1 | GsOYE |
|---|---|---|---|
| | (N = 4) | (N = 3) | (N = 3) |
| TON enzyme not | 520 ± 154 | 533 ± 145 | 860 ± 250 |
| immobilized (free) | | | |
| TON immobilized | 898 ± 87 | 916 ± 155 | 1624 ± 28 |
| enzyme | | | |
| Immobilized | 172 | 172 | 189 |
| enzyme/Free(%) | | | |
| Conversion rate with | 33 ± 5 | 75 ± 25 | 14 ± 2 |
| non-immobilized | | | |
| enzyme (free)(%) | | | |
| Conversion rate with | 37 ± 3 | 93 ± 9 | 52 ± 1 |
| immobilized | | | |
| enzyme (%) | | | |
| Immobilized | 112 | 124 | 371 |
| enzyme/Free(%) | | | |

⁽Trypanosoma brucei pyrophosphatase (TbVSP1, EC3 Hydrolase), transaminase B9L0N2 (w-TA, EC2 Transferase) and G. sulphuraria Old Yellow Enzyme (GsOYE, EC1 Oxydoreductase).

In all 3 cases, the TON of the immobilized enzyme is significantly higher than that of the non-immobilized enzyme. The conversions obtained with the immobilized enzymes are always equal to or greater than those obtained with the respective free enzymes.

An aerogel complexed with Co²⁺ ions obtained under the same conditions as those described above in the presence of Fe³⁺ ions and a B9L0N2 transaminase (w-TA, EC2 transferase) bearing a polyhistidine label at its N-terminal end was prepared. The ratio of TON immobilized enzyme to TON non-immobilized enzyme (free) is 764/520.

These results are better than those described to date in the literature where it is observed that in most cases the activity of the enzyme is greatly reduced after immobilization.

The interconnected macroporous structure (pores from 50 nm and 2 microns) therefore facilitates the mass transfer of reactants and products throughout the material.

### Example 5: Reuse

The enzymatic performance measured is expressed in the number of enzyme-catalyzed reactions (Turnover numbers, TON) over a given time. These activities are measured for both non-immobilized (free) enzymes and immobilized enzymes under the reaction conditions below.

For TbVSP1, the reaction studied is the hydrolysis of pyrophosphate to phosphate in the presence of the cofactor Mg²⁺. The protein load used is 10% by mass. The reaction mixture consists of sodium pyrophosphate (5 mM), MgCl2 (1 mM) in solution in Tris-HCl buffer 50 mM NaCl 200 mM pH 8.5. The solid consisting of 1 mg of TbVSP1 carrying a polyhistidine sequence immobilized on a hydrogel complexed with Fe³⁺ ions (10 mg) is added to the reaction mixture. The formation of the phosphate is monitored by determination of phosphomolybdate/malachite green in visible spectroscopy at 630 nm.

For w-TA, the reaction studied is the transamination of pyruvate in the presence of racemic α-methylbenzylamine to alanine and acetophenone in the presence of the pyridoxal phosphate cofactor (PLP). The protein load used is 10% by mass. The reaction mixture consists of pyruvate (25 mM), racemic α-methylbenzylamine (25 mM) and PLP (0.1 mM) and DMSO (1% wt) in solution of Phosphate buffer 50 mM NaCl 300 mM pH 7.5. The solid consisting of 1 mg of w-TA carrying a polyhistidine sequence immobilized on a hydrogel complexed with Fe³⁺ ions (10 mg) is added to the reaction mixture. The formation of acetophenone is continuously monitored by UV spectroscopy at 245 nm.

For GsOYE, the reaction studied is the reduction of cyclohexenone and cyclohexanone in the presence of the cofactor NADH which oxidizes to NAD+. The protein load used is 10% by mass. The reaction mixture consists of cyclohexenone (50 mM), NADH (75 mM) and DMSO (1% wt) in solution in Tris-HCl buffer 50 mM NaCl 150 mM pH 7.5. The solid consisting of 1 mg of GsOYE carrying a polyhistidine sequence immobilized on a hydrogel complexed with Fe³⁺ ions (10 mg) is added to the reaction mixture. The disappearance of NADH is monitored by UV spectroscopy at 340 nm.

Step 1: The solid consists of a protein carrying a polyhistidine sequence immobilized on a hydrogel complexed with Fe³⁺ ions is added to the reaction mixture.

Step 2: after 10 minutes of reaction at 25° C. for TbVSP1 and w-TA or after 1 hour of reaction at 30° C. for GsOYE, the suspension is centrifuged. The supernatant and the base are separated.

Step 3: The solid thus obtained is washed twice with a buffered aqueous solution: 1) Buffer is added (1 mL). 2) The suspension thus obtained is stirred for a few minutes (usually 5 minutes) at room temperature. 3) The suspension is centrifuged. 4) After centrifugation, the supernatant is removed. Steps 1 to 4 are repeated 1 time.

Steps 1, 2 and 3 are repeated successively 4 times, which corresponds to 5 cycles of use of the material consisting of a protein carrying a polyhistidine sequence immobilized on a hydrogel complexed with Fe³⁺ ions.

The results obtained are presented in Table 6.

TABLE 6
Catalytic performance (Turnover numbers, TON and Conversion
Rate) of the 3 immobilized enzymes studied

| | Cycle 1 | Cycle 2 | Cycle 3 | Cycle 4 | Cycle 5 |
|---|---|---|---|---|---|
| TONE w-TA | 898 ± 87 | 1041 ± 118 | 954 ± 1118 | 900 ± 133 | 838 ± 115 |
| TON | 533 ± 145 | 898 ± 140 | 779 ± 52 | 518 ± 135 | 383 ± 91 |
| TbVSP1 | | | | | |
| TON | 1624 ± 28 | 1504 ± 43 | 1340 ± 73 | 1195 ± 98 | 546 ± 88 |
| GsOYE | | | | | |

(Trypanosoma brucei pyrophosphatase (TbVSP1, EC3 Hydrolase), transaminase B9L0N2 (w-TA, EC2 Transferase) and G. sulphuraria Old Yellow Enzyme (GsOYE, EC1 Oxydoreductase) over 5 reaction cycles.

These results demonstrate that it is possible to use proteins carrying an immobilized polyhistidine sequence on a hydrogel complexed with Fe³⁺ ions at least 5 times in a row without significant loss of activity.

### Example 6: Leaching

For each reaction cycle, the three supernatants from steps 2 and 3 of the reuse protocol are kept (i.e. the reaction mixture after 10 min or 1 hour of reaction and the buffer solutions used for the two washes of the solid). The amount of protein contained in these solutions is measured by the Bradford method. The mass of protein contained in the solutions is thus obtained and related to the initial quantity of protein immobilized on the solid to obtain the leaching rate. The leaching rate for each cycle represents the amount of protein contained in the supernatant and in successive washes relative to the total amount of protein immobilized on the material. This rate is expressed in %.

The results are summarized in Table 7.

TABLE 7
Leaching rate measured at each cycle of use of the 3
immobilized enzymes studied of the 3 enzymes studied

| | Cycle 1 | Cycle 2 | Cycle 3 | Cycle 4 | Cycle 5 |
|---|---|---|---|---|---|
| Leaching w- | 0.9 ± 0.9 | 1.9 ± 2.1 | 1.8 ± 2.0 | 1.7 ± 2.0 | 0.3 ± 0.4 |
| TA | | | | | |
| Leaching | 3 ± 4 | 3 ± 4 | 2 ± 3 | 3 ± 4 | 2 ± 3 |
| TbVSP1 | | | | | |
| Leaching | 2 ± 0.7 | 2.1 ± 0.3 | 0.7 ± 0.2 | 0.4 ± 0.2 | 0.2 ± 0.1 |
| GsOYE | | | | | |

(Trypanosoma brucei pyrophosphatase (TbSVP1, EC3 Hydrolase), transaminase B9L0N2 (w-TA, EC2 Transferase) and G. sulphuraria Old Yellow Enzyme (GsOYE, EC1 Oxydoreductase) over 5 reaction cycles.

The leaching rates observed are very low. This means that the proteins adhere strongly to the surface of the materials studied. This observation is consistent with the strong affinity between the polyhistidine tag and the immobilized metal.

The observed leaching rates are very low: much lower than for other protein immobilization methods such as adsorption or encapsulation and of the same order of magnitude as for methods where a covalent bond to the support is formed via the use of a chemical grafting agent. However, we do not need the latter since the binding between the immobilized protein and the support is obtained thanks to the very high affinity between the immobilized metal and the protein carrying a polyhistidine tag. The metal itself interacts very strongly with the support via chemical functions on its surface (carboxylic acid, phenol, catechol in particular).

Flow Experiments (with and without Recirculation; without Recirculation=Continuous Flow)

### Recirculating Flow Using Enzyme B9L0N2 Immobilized on Fe-XCat

The reaction studied for the B9L0N2 enzyme is the transamination of pyruvate in the presence of racemic α-methylbenzylamine to alanine and acetophenone in the presence of pyridoxal phosphate cofactor (PLP). The reaction mixture consists of pyruvate (50 mM), racemic α-methylbenzylamine (50 mM), PLP (0.1 mM) and DMSO (1% wt) in solution in 50 mM NaCl phosphate buffer 300 mM pH=7.5.

The reaction mixture (10 mL) is introduced into the prepared column as described above in the “immobilization” section. The flow is recirculating, the solution recovered at the column outlet is directly reinjected into the column at a rate of 0.1 mL/min. A sample (100 μL) is taken every 10 minutes for 1 hour. The formation of acetophenone is monitored by UV spectrophotometry at 245 nm.

The enzyme activity measured at time t for immobilized B9L0N2 is expressed in mole of acetophenone formed per mole of enzyme. To do this, the amount of acetophenone is measured in each sample.

$${TON}_{t} = \frac{{[ acetophenone ]}_{t} \times V ( mL )}{{n}_{enzyme} ( μ mol )}$$

Conversion rates are expressed in moles of product formed (acetophenone) per moles of substrate (α-methylbenzylamine) initially present.

$$Conversion {rate}_{t} ( \% ) = \frac{{[ acetophenone ]}_{t} ( mM ) \times V ( mL )}{{[ α - methylbenzylamine ]}_{t = 0} ( mM ) \times V ( mL )} \times 100$$

### Continuous Flow Using the B9L0N2 Enzyme Immobilized on Fe-XCat

The reaction studied for the B9L0N2 enzyme is the transamination of pyruvate in the presence of racemic α-methylbenzylamine to alanine and acetophenone in the presence of pyridoxal phosphate cofactor (PLP). The reaction mixture consists of pyruvate (25 mM), racemic α-methylbenzylamine (25 mM), PLP (0.1 mM) and DMSO (1% wt) in solution in 50 mM NaCl phosphate buffer 300 mM pH=7.5.

The reaction mixture is introduced into the prepared column as described above in the “immobilization” section. The flow is continuous with a flow rate of 0.1 mL/min, the solution at the column outlet is recovered as a fraction of 1 mL for 5 hours. The formation of acetophenone is monitored by UV spectrophotometry at 245 nm.

The enzyme activity measured at time t for immobilized B9L0N2 is expressed in mole of acetophenone formed per mole of enzyme. To do this, the amount of acetophenone is measured in each sample.

$${TON}_{t} = \frac{{[ acetophenone ]}_{t} \times V ( mL )}{{n}_{enzyme} ( μ mol )}$$

Conversion rates are expressed in moles of product formed (acetophenone) per moles of substrate (α-methylbenzylamine) initially present.

$$Conversion {rate}_{t} ( \% ) = \frac{{[ acetophenone ]}_{t} ( mM ) \times V ( mL )}{{[ α - methylbenzylamine ]}_{t = 0} ( mM ) \times V ( mL )} \times 100$$

Flow with Recirculation Using the Enzyme GsOYE Immobilized on Fe-XCat

The reaction studied is the reduction of cyclohexenone to cyclohexanone in the presence of the cofactor NADH which oxidizes to NAD+. The reaction mixture consists of cyclohexenone (50 mM), NADH (50 mM) and DMSO (1% wt) in solution in Tris-HCl buffer 50 mM NaCl 150 mM pH=7.5.

The reaction mixture (10 mL) is introduced into the prepared column as described above in the “immobilization” section. The flow is recirculating, the solution recovered at the column outlet is directly reinjected into the column at a rate of 1 mL/min. A sample (100 μL) is taken every 10 min for 2 h (Day 1), then the column is stored overnight at 4° C. and a new reaction mixture (10 mL) is prepared to be reintroduced into the column (Day 2). The disappearance of NADH is monitored by UV spectrophotometry at 340 nm.

The enzyme activity measured for immobilized GsOYE is expressed in mole of NADH consumed per mole of GsOYE enzyme. To do this, the amount of residual NADH is measured in each sample.

$${TON}_{t} = \frac{( {[ NADH ]}_{t = 0} - {[ NADH ]}_{t} ) ( mM ) \times V ( mL )}{{n}_{enzyme} ( μ mol )}$$

Conversion rates are expressed in cofactor moles (NADH) consumed per initial cofactor moles (NADH).

$$Conversion {rate}_{t} ( \% ) = \frac{( {[ NADH ]}_{t = 0} - {[ NADH ]}_{t} ) ( mM ) \times V ( mL )}{{[ NADH ]}_{t = 0} ( mM ) \times V ( mL )} \times 100$$

### Results:

TABLE 8
Recirculating Flow Performance Using
Fe-XCat Immobilized B9L0N2 Enzyme

| Time (min) | Conversion rate (%) | TONE | Leaching (%) |
|---|---|---|---|
| 10 | 21.00 | 3062 | 0.00 |
| 20 | 23.22 | 3386 | 0.11 |
| 30 | 27.93 | 4074 | 0.32 |
| 40 | 33.55 | 4893 | 0.55 |
| 50 | 33.58 | 4897 | 0.55 |
| 60 | 33% | 4783.25634 | 0.55 |

TABLE 9
Non-recirculating (continuous flow) flow performance
using Fe-XCat immobilized B9LN02 enzyme

| Time (min) | Conversion rate (%) | TONE | Leaching % |
|---|---|---|---|
| 10 | 2.23 | 16 | 0.6 |
| 20 | 29.23 | 213 | 0.9 |
| 30 | 36.67 | 267 | 1.2 |
| 40 | 37.63 | 274 | 1.5 |
| 50 | 38.83 | 283 | 1.8 |
| 60 | 39.43 | 288 | 2.3 |
| 70 | 39.37 | 287 | 2.6 |
| 80 | 39.80 | 290 | 3.0 |
| 90 | 43.00 | 314 | 3.5 |
| 100 | 41.23 | 301 | 3.5 |
| 110 | 41.00 | 299 | 3.5 |
| 120 | 39.33 | 287 | 3.5 |
| 130 | 35.93 | 262 | 3.5 |
| 140 | 37.37 | 272 | 3.5 |
| 150 | 39.67 | 289 | 3.5 |
| 160 | 36.57 | 267 | 3.5 |
| 170 | 33.57 | 245 | 3.5 |
| 180 | 39.97 | 291 | 3.5 |
| 190 | 43.13 | 315 | 3.5 |
| 200 | 42.50 | 310 | 3.5 |
| 210 | 42.33 | 309 | 3.5 |
| 220 | 43.53 | 317 | 3.5 |
| 230 | 42.93 | 313 | 3.5 |
| 240 | 44.73 | 326 | 3.5 |
| 250 | 43.73 | 319 | 3.5 |
| 260 | 43.50 | 317 | 3.5 |
| 270 | 43.57 | 318 | 3.5 |
| 280 | 46.73 | 341 | 3.5 |
| 290 | 44.97 | 328 | 3.5 |
| 300 | 46.70 | 341 | 3.5 |

TABLE 10
Recirculating Flow Performance Using the
GsOYE Enzyme Immobilized on Fe-XCat

| | Time | Conversion rate (%) | TON (μmol/μmol) |
|---|---|---|---|
| Day 1 | 0 | 0 | 0 |
| | 10 | 4.03 | 133 |
| | 20 | 15.86 | 524 |
| | 30 | 23.26 | 768 |
| | 40 | 30.44 | 1005 |
| | 50 | 37.44 | 1236 |
| | 60 | 48.78 | 1611 |
| | 70 | 52.72 | 1741 |
| | 80 | 54.81 | 1810 |
| | 90 | 60.00 | 1620 |
| | 100 | 65.35 | 2158 |
| | 110 | 62.34 | 2058 |
| | 120 | 65.49 | 2162 |
| Day 2 | 120 | 0.00 | 0 |
| | 130 | 4.27 | 167 |
| | 140 | 5.73 | 224 |
| | 150 | 10.63 | 415 |
| | 160 | 14.04 | 549 |
| | 170 | 14.11 | 552 |
| | 180 | 28.23 | 1103 |
| | 190 | 31.19 | 1219 |
| | 200 | 35.94 | 1405 |
| | 210 | 40.06 | 1565 |
| | 220 | 45.90 | 1794 |
| | 230 | 51.78 | 2023 |
| | 240 | 57.88 | 2262 |

The experimental results with B9L0N2 in recirculating flow, in continuous flow (without recirculation) and with GsOYE in recirculating flow demonstrate the possibility of using the Fe-XCat material to immobilize different enzymes and use them in flow. The low leaching rates (<5%) indicate that the enzymes remain overwhelmingly interacting with the immobilizing support material.

1-10. (canceled)

11. A composition comprising:
a porous carbon-based material obtained by hydrothermal carbonization comprising or formed from:
at least one ose, or at least one polyoside,
at least one compound having an orthodiphenol or orthomethoxyphenol function, and
at least one metal cation,
said porous carbon-based material has at least one orthodiphenol function.

12. The composition of claim 11, wherein,
the ose is:
an aldose chosen from: glyceraldehyde, erythrose, threose, ribose, arabinose, xylose, lyxose, allose, altrose, glucose, mannose, gulose, idose, galactose, and talose, or
a ketose chosen from: dihydroxyacetone, erythrulose, ribulose, xylulose, fructose, psicose, sorbose, and tagatose, or
the polyoside is:
a heterodiholoside chosen from: trehalulose, sucrose, turanose, maltulose, leucrose, isomaltulose, gentiobiulose, melidiose, lactulose, lactose, and rutinose, or
a homodiholoside chosen from: inulobiose, alpha2-mannobiose, alpha3-mannobiose, trehalose, kojibiose, nigerose, maltose, isomaltose, sophorose, laminaribiose, cellobiose, and gentiobiose.

13. The composition of claim 11, wherein the compound having an orthodiphenol or orthomethoxyphenol function is selected from: catechol, pyrogallol, caffeic acid, mangiferin, quercetin, cyanidin, catechin, epicatechin, epigallocatechin, anthocyanidol, procyanidol B-3, procyanidol B-4, fuhalols (bifuhahol, trifuhahol), carmalol (diphlorethohydroxycamalol) and tannic acid, or a natural vegetable extract containing at least one of these compounds.

14. The composition of claim 13, wherein the compound having an orthodiphenol function is catechin.

15. The composition of claim 11, wherein the porous carbon material is formed from:
maltose, galactose, lactose, glucose, mannose, sucrose, arabinose, ribose, fructose, xylose, erythrose, dihydroxyacetone, and
catechin, or an extract of catechu.

16. The composition of claim 11, wherein the porous carbon-based material has a molar ratio between the at least one ose, or at least one polysaccharide and the at least one compound having at least an orthodiphenol or orthomethoxyphenol function of 1/2 or 1/1 or 2/1 or 3/1 or4/1 or 5/1.

17. The composition of claim 11, wherein the at least one metal cation is selected from Al³⁺, Ag⁺, Ba²⁺, Ca²⁺, Cd²⁺, Co²⁺, Cr³⁺, Cu⁺, Cu²⁺, Fe²⁺, Fe³⁺, K⁺, Mg²⁺, Mn²⁺, Na⁺, Ni²⁺, Pb²⁺, Sn²⁺, Sn⁴⁺, Zn²⁺.

18. The composition of claim 11, further comprising at least one immobilized protein.

19. The composition of claim 11, wherein an amount of protein adsorbed on the porous carbon material is from 10 to 80 parts by weight in relation to 100 parts by weight of the porous carbon material.

20. The composition of claim 18, wherein the specific activity of the immobilized enzyme on the porous carbon-based material is greater than that of the same free enzyme.

21. The composition of claim 18, wherein said at least one immobilized protein comprises a poly-histidine label.

22. The composition of claim 18, wherein said at least one immobilized protein is an enoate reductase (EC 1.3.1.31), a transaminase (EC 2.6.1), or a pyrophosphatase (EC3.6.1.1).
